(19) United States
(12) Patent Application Publication
(10) Pub. No.: US 2017/0296748 A1
(43) Pub. Date: Oct. 19, 2017

(54) Torsion Spring for an Injection Device and an Injection Device Comprising Such Torsion Spring

(71) Applicant: NOVO NORDISK A/S, Bagsvaerd (DK)

(72) Inventor: Soeren Dybdal REIMER, Birkeroed (DK)

(21) Appl. No.: 15/517,051

(22) PCT Filed: Oct. 7, 2015
(86) PCT No.: PCT/EP2015/073105

§ 371 Date: Apr. 5, 2017

(30) Foreign Application Priority Data
Oct. 8, 2014 (EP) 14188136.7

(51) Int. Cl. A61M 5/20; A61M 5/315

(52) U.S. Cl. CPC A61M 5/20; A61M 5/31553; A61M 5/31583

(57) ABSTRACT

The present invention relates to a helically coiled torsion spring for a torsion spring based automatic injection device, coiled in a longitudinal direction (X) and having a number of consecutive windings located between a distal winding having a distal end and a proximal winding having a proximal end. Each winding further has an outwardly pointing surface. At least one end of the torsion spring is abruptly cut to form a flat end surface with no bends and at least a number of the consecutive windings are coiled with a gap between the consecutive windings such that that the torsion spring apply an axial force when the distal end and the proximal end are moved axially against each other. The invention further relates to a torsion spring based automatic injection device for expelling settable doses of a liquid drug utilizing such torsion spring to urge the abruptly cut ends into proper engagement.

### THE TECHNICAL FIELD OF THE INVENTION

The invention relates to helically coiled torsion springs and to torsion spring based automatic injection devices utilizing such torsion springs. The invention especially relates to securing such helically coiled torsion spring to polymeric parts in the automatic injection device. Most especially the invention relates to helically coiled torsion springs having no bend at their respective end and thus is strained by pushing the respective non-bended ends relatively against each other.

### DESCRIPTION OF RELATED ART

Automatic injection devices in which a user strains a torsion spring during dose setting and wherein the torque stored in the torsion spring is utilized to expel the liquid drug has been known for decades. An early example of such automatic injection device for expelling settable dose sizes is e.g. provided in U.S. Pat. No. 5,104,380.

The torsion springs used in such automatic injection devices are usually helically coiled torsion springs. The helical torsion spring is usually positioned between the housing and a rotatable dose setting member and strained by rotating the dose setting member. WO2006/045526 discloses a helical torsion spring in which the distal end has an outwardly bend for securing the torsion spring to the housing and the proximal end has an inwardly bend for attaching the torsion spring to the rotatable dose setting member.

The same is the case for WO2007/063342 in which the helical torsion spring distally is provided with a hook-like bend engaging the housing via a retaining ring moulded integrally with the housing and proximally has an inwardly bend that engages the ratchet drive shaft which is rotatable secured to the dose setting knob. Thus, when a user rotates the dose setting knob the torsion spring is strained.

The helical torsion spring is further disclosed in WO 2012/063061 which discloses a number of examples on how the bended end of the helical torsion spring can be secured to parts of the injection device.

Further, torsion springs for spring based automatic injection devices having bended ends and coiled in an open coil form is disclosed in WO 2006/126902, WO2010/089418 and in WO 2013/167869.

When used in an automatic injection device the torsion spring is usually build into the construction such that the axial length of the torsion spring do not change when a torque is being build up in the torsion spring as no part grows out of the injection device during dose setting. Both bended ends are secured in the injection device in axially fixed positions and rotational twisted away from each other relatively during dose setting which makes the diameter of the torsion spring decrease during dose setting.

When producing helical coiled torsion springs it is associated with additional costs to make bends at the end of the helical torsion spring. Helical torsion springs having bended ends are thus more expensive than helical torsion springs without bended ends. It would thus be beneficial if a helical torsion spring without bends could be used in an automatic injection device. Examples of an injection device having a torsion spring with abruptly cut ends forming straight and non-bended end surfaces are provided in WO 2014/001318 and in WO 2014/060369.

When a dose is set in such injection device the straight ends of the torsion spring is rotational twisted in a direction against each other relatively which increases the diameter of the helical torsion spring. Also here are both ends secured in axially fixed positions in the injection device such that the length of the torsion spring remains constant.

Further, if a user selects only a small dose to be injected, a certain torque needs to be available in the torsion spring in order to deliver sufficient force to expel such small dose. The force must be sufficient to overcome the friction in the dose mechanism. This requires that the torsion spring is pre-strained during manufacture of the injection device such that a certain torque is present in the torsion spring even when no dose has been selected i.e. when the dose setting mechanism is in its “zero” position. Only by having a pre-strained torsion spring is there sufficient torque to overcome the friction in the dose mechanism and expel a small dose. Mathematically, the “zero” position of the dose setting mechanism have to be positioned a certain distance up on the spring characteristic of the torsion spring such that the torsion spring already applies a certain torque in this “zero” position.

Usually both the distal end of the torsion spring and the proximal end of the torsion spring (or at least one of the ends) are secured to polymer components in the injection device as disclosed in WO 2014/001318. This however creates a problem when operating with pre-strained torsion springs because the torque loaded in the torsion spring applies stress to the polymeric parts securing the torsion spring which make the polymer creep over time. It is especially a problem when using torsion springs with no bends since the area on the polymeric part that the spring acts upon is limited to the diameter of the wire from which the torsion spring is coiled. Further, such automatic injection devices are often stored for a substantial period of time and sometime under changing temperature condition which further exposes the polymer components under stress from the pre-tensed spring to crack propagation.

Automatic injection devices having pre-strained torsion springs therefore need to be designed such that the torque of the torsion spring is obtained by the polymeric part over a substantial area thereby reducing the stress on the polymeric parts.

Further, it is desirable if the abruptly cut ends of the torsion spring abuts the polymeric parts in a specific position independently on the tolerances of the polymeric parts.

### DESCRIPTION OF THE INVENTION

It is an object of the present invention to provide an automatic torsion spring driven injection device for apportioning settable doses of a liquid drug and wherein the torsion spring has no bends at least at one end thereby reducing production costs. Further, it is an object to provide a way of mounting a torsion spring reducing stress on the polymeric parts securing the torsion spring. The reduction of stress can be understood to be the stress occurring during dose setting i.e. when straining the torsion spring or it can be the stress applied by a pre-strained torsion spring during storing of the injection device or it can be either in combination.

It is a further object to provide a low cost torsion spring having at least one abruptly cut end forming a straight and non-bended end surface wherein the spring end surfaces automatically position themselves in a specific position, especially in the axial direction, in each individual injection device during assembly of the device. This automatic positioning of the spring end surfaces should preferably happen independently of the tolerances of the components making up the individual injection device.

By a cut end surface having no bends is meant that the wire forming the torsion spring at one end is abruptly cut in a direction predominantly perpendicular to the length of the wire. The end of the spring thus forms a predominantly straight configuration which is not bended and which follows the radius of the coiled torsion spring.

The invention is defined in claim 1. Accordingly in one aspect, the present invention relates to a helically coiled torsion spring comprising a number of consecutive windings wherein a distal winding has a distal end and a proximal winding has a proximal end, and each winding further has an outwardly pointing surface. One (or both) of the distal end and the proximal end are cut in a direction mainly perpendicular to the length of the coiled wire to form a predominantly straight and non-bended end surface. Further, a number of the consecutive windings in a first region are coiled with no gap between the consecutive windings to form a closed coil form and a number of the consecutive windings in a second region are coiled with a gap between the consecutive windings to form an open coil form such that that the torsion spring apply an axial force when the distal end and the proximal end are moved axially against each other

By having a compression region coiled in an open form, the distal end and the proximal end are urged away from each other and urged against their respective supporting surfaces independently on the tolerances of the various parts making up the spring based injection device. The torsion spring is thus self-aligning in an axial direction and do not depend on a very precise length of the space in which the torsion spring is built in thus allowing relatively large tolerances.

The number of windings coiled with a gap in the second region can be any number depending on the axial force required to urge the spring ends in their respective positions. The second region coiled in an open form is preferably located next to a first region wherein the windings are coiled in a closed form. The torsion spring can comprise any random number of first and second regions located in any random position, but are preferably formed by two first regions coiled in a closed form separated by one second region coiled in an open form.

In a second aspect, the invention relates to a torsion spring based automatic injection device utilizing the helically coiled torsion spring. Such torsion spring based injection device comprises a housing assembly and a dose setting assembly which is rotatable relatively to each other to set a dose. The torsion spring is encompassed between the housing assembly and the dose setting assembly such that the torsion spring is strained whenever the dose setting assembly is rotated relatively to the housing assembly.

The torsion spring is a helically coiled torsion spring having a longitudinal direction and a number of consecutive windings wherein a distal winding has a distal end and a proximal winding has a proximal end. One or both of these ends are abruptly cut to form a flat end surface preferably having the same (or approximately the same diameter) as the wire from which the torsion spring is coiled. Each of the windings including the distal and the proximal winding has an outwardly pointing surface.

Further, a number of the consecutive windings are coiled with a gap between the consecutive windings such that the torsion spring applies an axial force when the distal end and the proximal end are moved against each other.

In a pen-shaped automatic injection device, the helically coiled torsion spring and the injection device follows the same centre line and the two ends of the torsion spring is urged apart into correct abutment with the two surfaces pressing on the abruptly cut ends of the torsion spring.

At least a part of the housing assembly or a part the dose setting assembly is made from a polymeric material and comprises a spring receiving arrangement which is made from a polymeric material and comprises a first surface extending substantially parallel with the longitudinal direction of the helical torsion spring for abutting the abruptly cut flat end of the distal or the proximal end of the helical torsion spring and which spring receiving arrangement further comprises a second surface substantially parallel with the longitudinal direction of the helical torsion spring for supporting the outwardly pointing surface of the at least distal winding or the at least proximal winding.

As a result when the distal end and the proximal end of the helical torsion spring is twisted towards each other by abutment with the first surface, the outside diameter of the helical torsion spring increases and the outer surface of the torsion spring will abut with the second surface. During this abutment some of the torque built up in the helical torsion spring will be transmitted as friction against this second surface thereby releasing the first surface from some stress.

Either the distal end or the proximal end of the helical spring (or both ends) are abruptly cut to form flat end surfaces. By abruptly cut means that the wire forming the torsion spring is cut over in a direction substantially perpendicular to its length. However, the cut ends could be bended and pressed together in order to stiffen the abutment with the housing assembly and/or the dose setting assembly. The important feature being that the first surface of the spring receiving arrangement pushes on the end surface of the torsion spring when increasing the dose size.

The dose setting assembly comprises a dose setting member and the housing element comprises a housing member. One or both of these members has an integrally formed spring receiving arrangement for receiving one or both ends of the helical coiled torsion spring encompassed between the dose setting member and the housing member.

The dose setting member and the housing member is preferably arranged in a permanent axial distance and maintained in that permanent axial distance during dose setting and dose expelling thus the helical coiled torsion spring maintains its axial length during operation of the automatic injection device.

Further, the spring receiving arrangement comprises a cut-out. However, the spring receiving arrangement is not necessarily physically cut into the housing member and/or the dose setting member. The spring receiving arrangement including the cut-out is preferably formed from a polymeric material in a moulding process.

The cut out generates the first surface which one or both ends of the helical coiled torsion spring abuts.

In one embodiment a guiding surface is provided for guiding the end of the helical coiled torsion spring into abutment with the first surface. This guiding surface preferably extend in a direction substantially perpendicular to the longitudinal direction of the helical coiled torsion spring such that it intercepts the spring next to the end of the spring and lifts the end into position.

The second surface which is also in parallel with both the longitudinal direction of the helical coiled torsion spring and the first surface has in one embodiment a step-wise configuration with each step having an extension substantial equal to the diameter of the spring wire to support each winding. Each step can tilt a few degrees inwardly to provide a better grip with each windings.

### Definitions

An “injection pen” is typically an injection apparatus having an oblong or elongated shape somewhat like a fountain pen for writing. Although such pens usually have a tubular cross-section, they could easily have a different cross-section such as triangular, rectangular or square or any variation around these geometries.

An injection pen is often referred to as being either “Pre-filled” or “durable”. By the term “Pre-filled” injection device is meant an injection device in which the cartridge containing the liquid drug is permanently embedded in the injection device such that it cannot be removed without permanent destruction of the injection device. Once the pre-filled amount of liquid drug in the cartridge is used, the user normally discards the entire injection device. This is in opposition to a “Durable” injection device in which the user can himself change the cartridge containing the liquid drug whenever it is empty. Pre-filled injection devices are usually sold in packages containing more than one injection device whereas durable injection devices are usually sold one at a time. When using pre-filled injection devices an average user might require as many as 50 to 100 injection devices per year whereas when using durable injection devices one single injection device could last for several years, however, the average user would require 50 to 100 new cartridges per year.

Further, using the term “Automatic” in conjunction with injection device means that, the injection device is able to perform the injection without the user of the injection device delivering the force needed to expel the drug during dosing. The force is typically delivered—automatically—by an electric motor or by a spring drive driving the set dose out from the cartridge through the lumen of the needle cannula and into the skin of the user. The spring for the spring drive can be any kind of spring, including torsion springs, and is usually strained by the user during dose setting. The spring is preferably prestrained in order to avoid problems of delivering very small doses. Alternatively, the spring can be fully preloaded by the manufacturer with a preload sufficient to empty the entire drug cartridge though a number of doses. Typically, the user activates a latch mechanism e.g. in the form of a button on the injection device, e.g. on the proximal end of the injection device to release—fully or partially—the force accumulated in the spring when carrying out the injection.

“Cartridge” is the term used to describe the container containing the drug. Cartridges are usually made from glass but could also be moulded from any suitable polymer. A cartridge or ampoule is preferably sealed at one end by a pierceable membrane referred to as the “septum” which can be pierced e.g. by the non-patient end of a needle cannula. Such septum is usually self-sealing which means that the opening created during penetration seals automatically by the inherent resiliency once the needle cannula is removed from the septum. The opposite end is typically closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug. However, any kind of container—rigid or flexible—can be used to contain the drug.

Since a cartridge usually has a narrower distal neck portion into which the plunger cannot be moved not all of the liquid drug contained inside the cartridge can actually be expelled. The term “initial quantum” or “substantially used” therefore refers to the injectable content contained in the cartridge and thus not necessarily to the entire content.

The term “Needle Cannula” is used to describe the actual conduit performing the penetration of the skin during injection. A needle cannula is usually made from a metallic material such as e.g. stainless steel and connected to a hub to form a complete injection needle also often referred to as a “needle assembly”. A needle cannula could however also be made from a polymeric material or a glass material. The hub also carries the connecting means for connecting the needle assembly to an injection apparatus and is usually moulded from a suitable thermoplastic material. The “connection means” could as examples be a luer coupiing, a bayonet coupling, a threaded connection or any combination thereof e.g. a combination as described in EP 1,536,854.

As used herein, the term “drug” is meant to encompass any drug-containing flowable medicine capable of being passed through a delivery means such as a hollow needle in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

The invention will be explained more fully below in connection with a preferred embodiment and with reference to the drawings in which:

FIG. 1A show a cross sectional view of the torsion spring arrangement.

FIG. 1B show a cut-over perspective view of the torsion spring arrangement of FIG. 1A.

FIG. 2A-B show cross sectional views (180 degrees displaced) of the torsion spring attachment with the housing member.

FIG. 2C show a cut-over perspective view of the torsion spring attachment with the housing member.

FIG. 3A-B show cross sectional views (180 degrees displaced) of the housing member.

FIG. 3C show a cut-over exploded view of the housing member.

FIG. 4A-B show cross sectional views (180 degrees displaced) of the alternative torsion spring attachment with the housing member.

FIG. 4C show a cut-over perspective view of the torsion spring attachment with the housing member.

FIG. 5A-B show cross sectional views (180 degrees displaced) of the alternative housing member.

FIG. 5C show a cut-over exploded view of the alternative housing member.

FIG. 6 show a different embodiment of the torsion spring.

FIG. 7 show an enlarged cross sectional view of the embodiment depicted in FIG. 6.

The figures are schematic and simplified for clarity, and they just show details, which are essential to the understanding of the invention, while other details are left out. Throughout, the same reference numerals are used for identical or corresponding parts.

### Detailed Description of Embodiment

When in the following terms as “upper” and “lower”, “right” and “left”, “horizontal” and “vertical”, “clockwise” and “counter clockwise” or similar relative expressions are used, these only refer to the appended figures and not to an actual situation of use. The shown figures are schematic representations for which reason the configuration of the different structures as well as there relative dimensions are intended to serve illustrative purposes only.

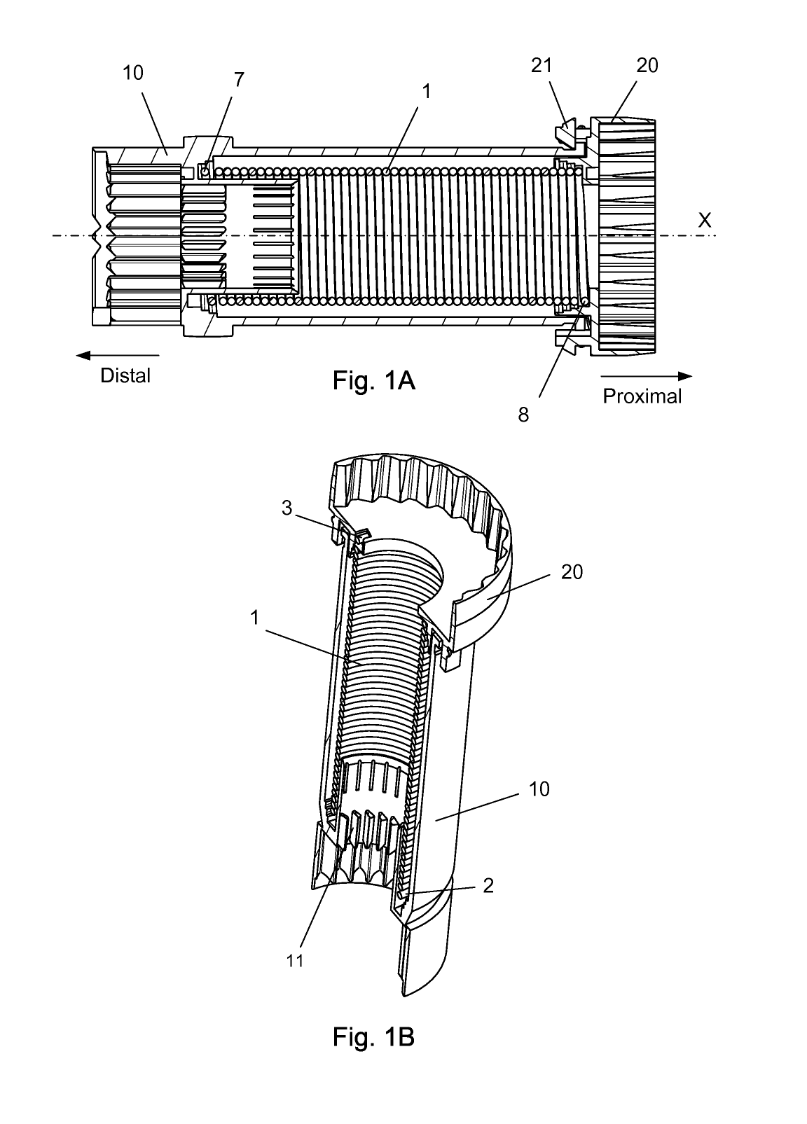

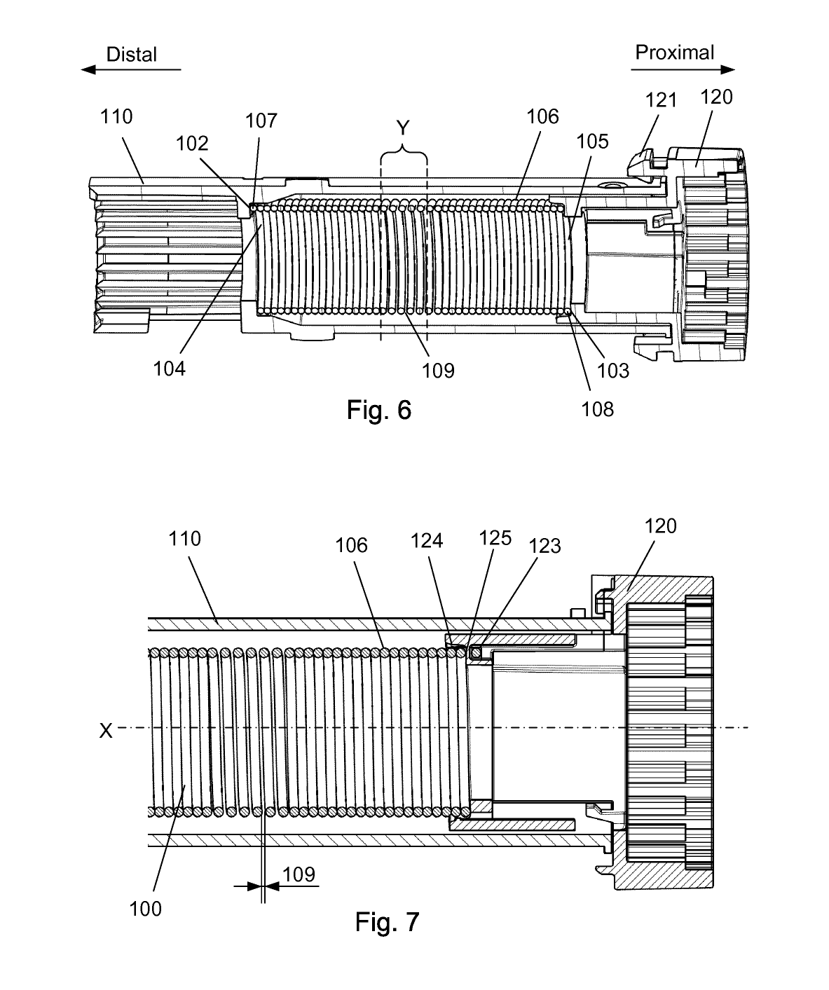

In that context it may be convenient to define that the term “distal end” in the appended figures is meant to refer to the end of the injection device which usually carries the injection needle whereas the term “proximal end” is meant to refer to the opposite end pointing away from the injection needle and usually carrying the dose dial button. The directions are indicated with arrows in FIGS. 1A and 1n FIG. 6.

FIG. 1A-B discloses a part of a torsion spring driven injection device according to a first embodiment of the invention. The torsion spring 1 is at its distal end 2 attached to a dose setting member 10 being a part of the dose setting assembly and at its proximal end 3 connected to a housing member 20 being part of the housing assembly.

The dose setting member 10 is further connected to a not-shown dose setting button via a toothed interface 11 such that the dose setting member 10 can be rotated when the user dials a dose. The housing member 20 is via locking protrusions 21 rotational locked to the not-shown housing but could alternatively be moulded integrally with the housing.

In WO 2014/001318 by Novo Nordisk A/S, which is incorporated by reference, the housing member (referred to as the spring base in FIG. 20) is numbered “180” and the dose setting member (referred to as the drive tube) is numbered “170”. The dose setting member“170” is connected to a distally located dose setting button (numbered “1004) via a ratchet element “185”. A scale drum “160” is slidable connected to dose setting member “170”. In the present invention a scale drum carrying indicia can be axially slidable connected to the dose setting member 10 which again is part of the dose setting assembly.

Whenever the user dials a dose by rotating the dose setting button, the dose setting member 10 rotates with it thereby straining the torsion spring 1 encompassed between the dose setting member 10 and the housing member 20.

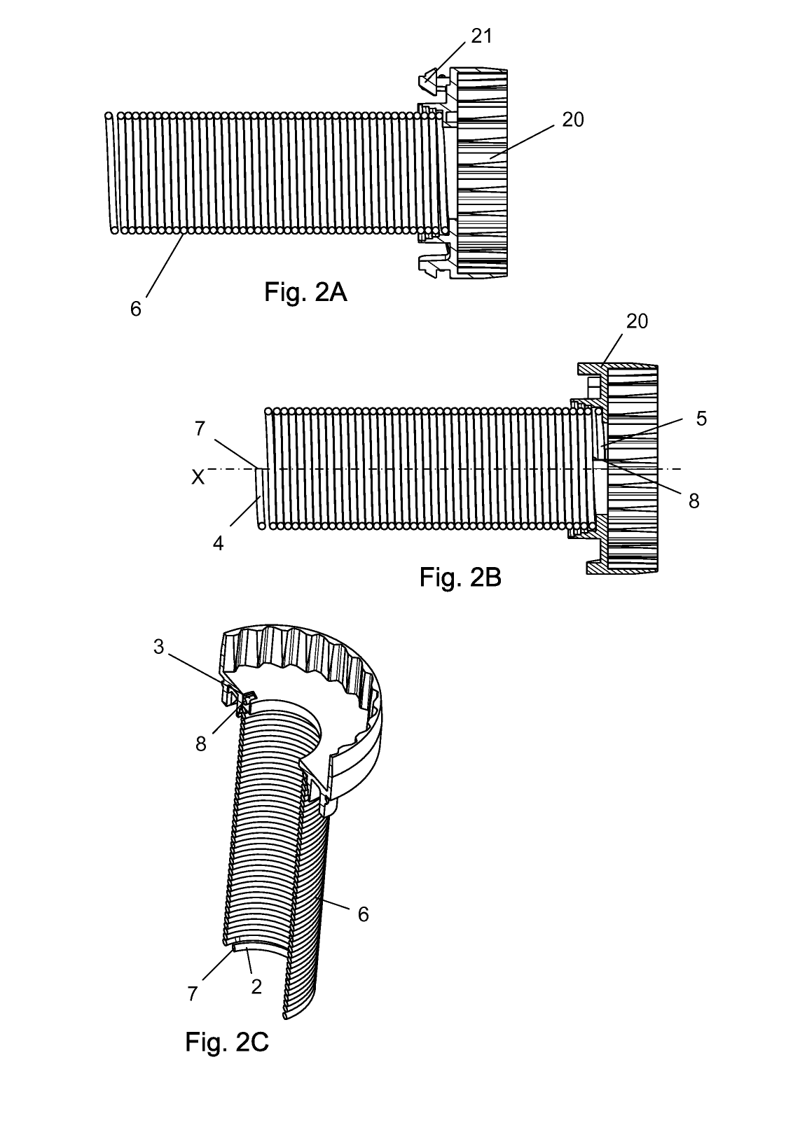

The connection between the housing member 20 and the torsion spring 1 is further disclosed in the FIGS. 2A-C, 3A-C and 4A-C.

The torsion spring 1 is helical coiled and has a distal winding 4 ending in a distal end 2 and a proximal winding 5 ending in a proximal end 3. Both these ends 2, 3 are abruptly cut to form flat end surfaces 7, 8 which are best seen at the distal end 2 in FIGS. 2B and 2C.

Further, as disclosed in FIG. 2C, each winding of the torsion spring 1 has an outer surface 6. Since the torsion spring 1 is coiled from a circular wire, the outer surface 6 runs in parallel with the longitudinal direction (X) of the helical spring 1 which is also the longitudinal direction of the injection device.

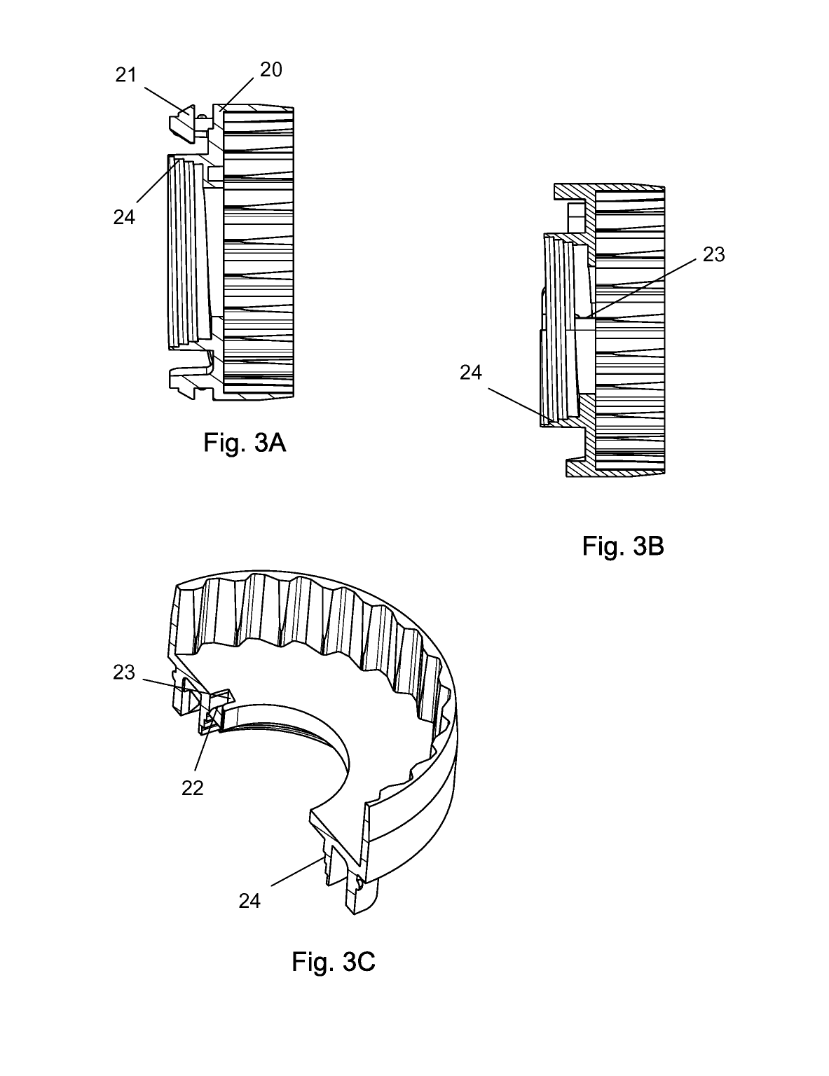

The spring receiving arrangement of the housing member 20 is further shown in the FIGS. 3A to 5C. A similar spring receiving arrangement can be provided in the dose setting member 10 as indicated in FIG. 1A-1B.

The arrangement has a cut-out 22 having a first surface 23 which is parallel to the longitudinal axis X of the torsion spring 1 such that the abruptly cut proximal surface 8 of the torsion spring 1 abuts this first surface 23 when the user strains the torsion spring 1.

Distally, the housing member 20 is provided with a second surface 24 also being in parallel with the longitudinal direction X of the torsion spring 1.

When the torsion spring 1 is strained by rotating the dose setting member 10 relatively to the housing member 20, the proximal flat end surface 8 abuts the first surface 23 and further rotation of the dose setting member 10 causes the outer diameter of the torsion spring 1 to be increased.

Since the torsion spring 1 has both flat end surfaces 7, 8 encompassed between two similar first surfaces 23 (the other surface is the not-shown first surface of the dose setting member 10) provided in the same permanent axial distance, the diameter of the torsion spring 1 will increase as the two surfaces 23 rotate relatively to each other building up torque in the torsion spring 1.

This increase of the outer diameter of the torsion spring 1 causes the outer surface 6 of at least the proximal winding 5 to abut the second surface 24 of the housing member 20.

The friction occurring between the outer surface 6 of the torsion spring 1 and the second surface 24 means that the torque build up in the torsion spring 1 during straining is distributed to the housing member 20 over a large area whereby stressing of the first surface 23 is minimized.

The second surface 24 has in one embodiment a stepwise configuration as best seen in FIG. 1A wherein each step is configured to abut the outer surface 6 of consecutively windings.

Each step of the second surface 24 can alternatively tilt inwardly towards the centreline X with a small angle which would provide a better grip on each consecutive winding.

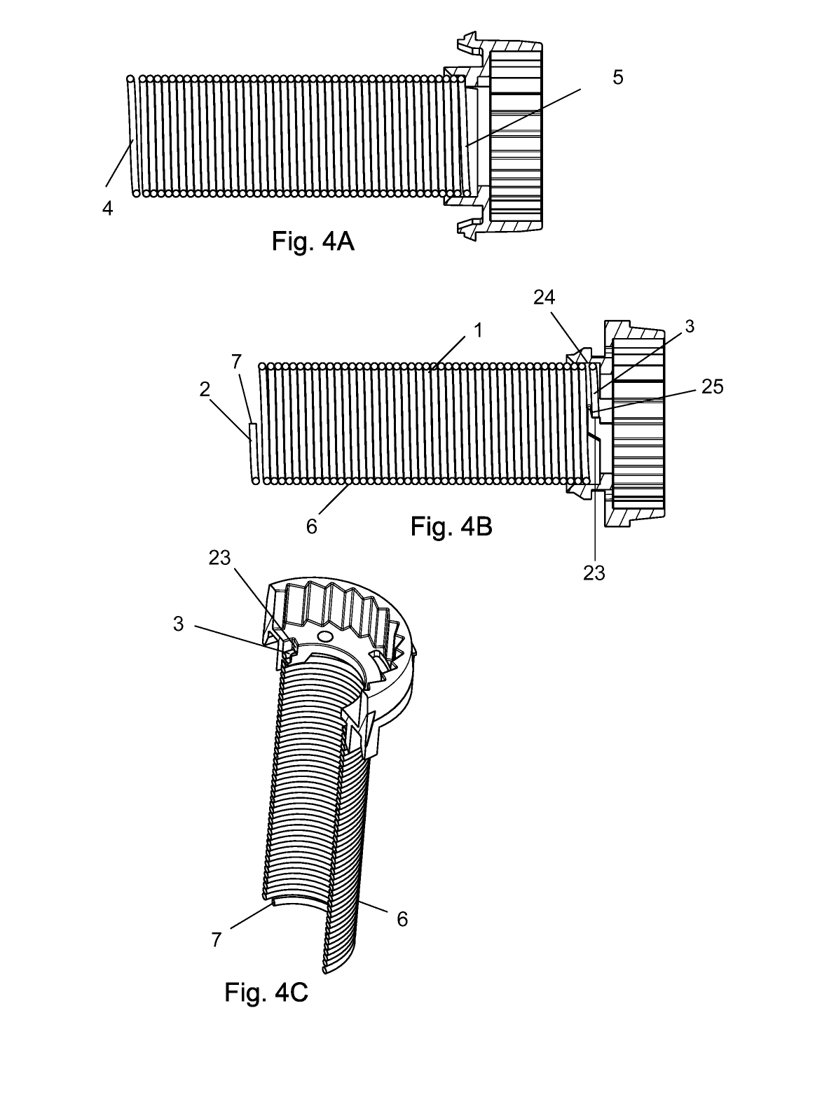

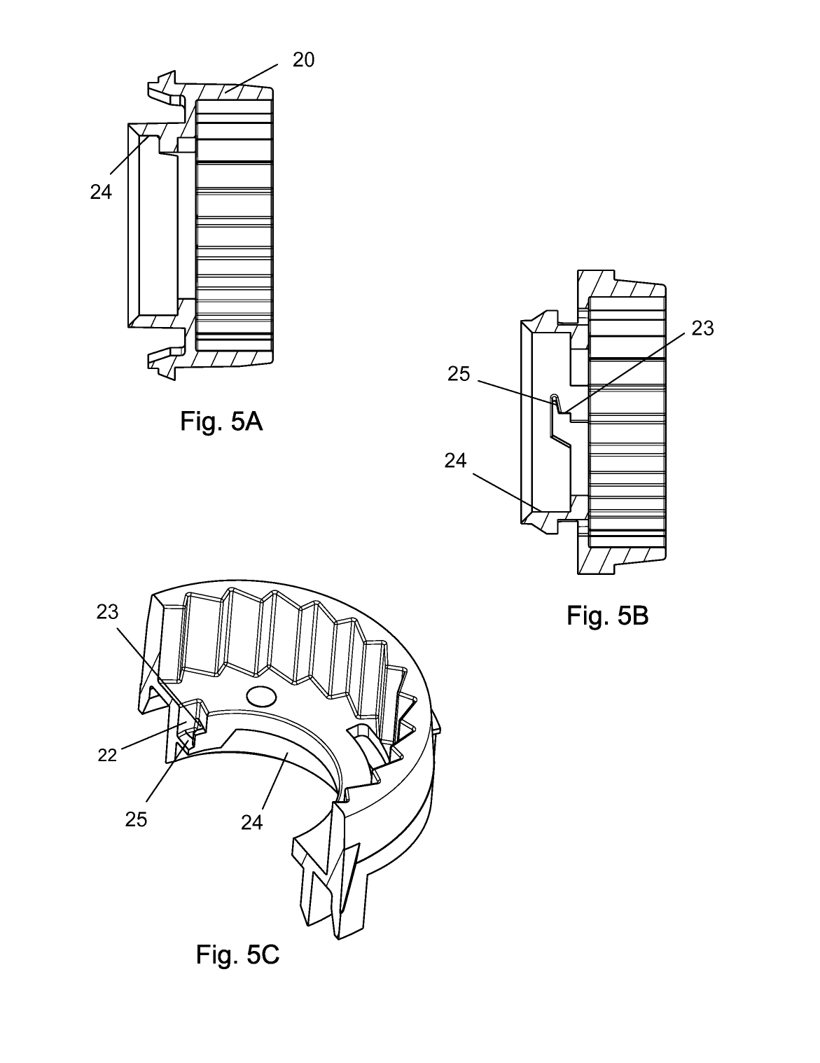

FIG. 4A-C and FIG. 5A-C discloses an alternative embodiment wherein the second surface 24 is parallel to the longitudinal extension (X) of the helical torsion spring (1) without any steps.

Also, as best seen in FIG. 5B and FIG. 5C, the cut-out 22 is provided with a distally located guiding surface 25 for guiding the abruptly cut flat surfaces 7, 8 of the torsion spring 1 into abutment with the first surface 23.

Further, as indicated in FIG. 1A-1B, the dose setting member 10 can be formed in the same way such that the torsion spring 1 is fixated in the same manner both in its distal end 2 and in its proximal end 3.

FIGS. 6 and 7 discloses a different embodiment in which the torsion spring 100 is also encompassed between a housing assembly and a dose setting assembly.

The dose setting assembly comprises a dose setting member 110 being connected to a not-shown dose setting button via the toothed interface 111 as in the previous embodiment.

The housing assembly comprises a housing member 120 connected the housing assembly via a number of protrusions 121.

This torsion spring 100 is also at a distal end 102 provided with a distal winding 104 having an abruptly cut end surface 107 and the proximal end 103 has a proximal winding 105 with an abruptly cut end surface 108.

The distally abruptly cut end surface 107 abuts the dose setting member 110 and the proximally located abruptly cut end surface 108 abuts the housing member 112 such that the torsion spring 100 is strained when the dose setting member 110 is rotated relatively to the dose setting member 120 during dose setting.

The housing member 120 is, as in the first embodiment, provided with a second surface 124 which is parallel with the longitudinal direction X of the torsion spring 100. This is e.g. disclosed in FIG. 7 which depicts an enlarged view of the proximal end.

The outwardly pointing surface 106 of at least the proximal winding 105 at the proximal end 103 is thus forced against this second surface 124 whenever the dose setting member 110 is rotated to set a dose.

A similar design is preferably but not necessarily provided at the distal end 102 of the torsion spring 100.

Further, the torsion spring 100 of the second embodiment is provided with a zone or region Y in which the coils of the torsion spring 100 is coiled with a gap 109 between consecutive windings.

Due to the gap 109, the torsion spring 100 applies an axial force when compressed such that the abruptly cut surface 107 (when viewing the proximal end as in FIG. 7) during assembly is in a position in which the guiding surface 125 can properly grip the proximal end 103 of the torsion spring 100 and guide the abruptly cut surface 108 into abutment with the first surface 123 (23 in the embodiment in FIG. 5C).

The axial force urges the distal end 102 and the proximal end 103 away from each other and into proper engagement with their respective guiding surfaces (125 for the proximal end 103 in FIG. 7). In this position, the first surfaces (123 for the proximal end 103) pushes properly on the abruptly cut end 108 (107 for the distal end).

Some preferred embodiments have been shown in the foregoing, but it should be stressed that the invention is not limited to these, but may be embodied in other ways within the subject matter defined in the following claims.

1. A helically coiled torsion spring in a torsion spring based automatic injection device, coiled in a longitudinal direction and comprising:
a number of consecutive windings wherein a distal winding has a distal end and a proximal winding has a proximal end, and each winding further having an outwardly pointing surface, wherein
one or both of the distal end and the proximal end are cut to form a predominantly straight and non-bended end surface, and wherein
a number of the consecutive windings in a first region are coiled with no gap between the consecutive windings to form a closed coil form and a number of the consecutive windings in a second region are coiled with a gap between the consecutive windings to form an open coil form such that that the torsion spring apply an axial force when the distal end and the proximal end are moved axially against each other.

2. A torsion spring based automatic injection device for expelling settable doses of a liquid drug comprising:
a housing assembly and a dose setting assembly being relatively rotatable,
a torsion spring according to claim 1 encompassed between the housing assembly and the dose setting assembly such that the torsion spring is strained upon rotation of the dose setting assembly relatively to the housing assembly, and wherein
the torsion spring is helically coiled having a longitudinal direction and a number of consecutive windings wherein a distal winding has a distal end and a proximal winding has a proximal end, the distal end and/or the proximal end are cut to form a straight and non-bended end surface, each winding further having an outwardly pointing surface, wherein a number of the consecutive windings in a first region are coiled with no gap between the consecutive windings to form a closed coil form and a number of the consecutive windings in a second region are coiled with a gap between the consecutive windings to form an open coil form such that that the torsion spring applies an axial force when the distal end and the proximal end are moved axially against each other, and wherein
at least one of the housing assembly or the dose setting assembly is at least partly made from a polymeric material and comprises a spring receiving arrangement comprising a first surface substantially parallel with the longitudinal direction of the helical torsion spring for abutting the distal end surface or the proximal end surface of the helical torsion spring and which spring receiving arrangement further comprises a second surface substantially parallel with the longitudinal direction of the helical torsion spring for supporting the outwardly pointing surface of the at least distal winding or the at least proximal winding,
and wherein when the dose setting assembly are rotated relatively to the housing assembly to strain the torsion spring, the first surface of the spring receiving arrangement is pressed against the cut, straight and non-bended end surface of the torsion spring to strain the torsion spring thereby forcing the outwardly pointing surface of the at least distal winding and/or the at least proximal winding to press against the second surface.

3. A torsion spring based automatic injection device according to claim 2, wherein the dose setting assembly comprises a polymeric dose setting member in which the spring receiving arrangement is integrally formed.

4. A torsion spring based automatic injection device according to claim 2, wherein the housing assembly comprises a polymeric housing member in which the spring receiving arrangement is integrally formed.

5. A torsion spring based automatic injection device according to claim 2, wherein the spring receiving arrangement comprises a cut-out.

6. A torsion spring based automatic injection device according to claim 5, wherein the first surface is part of the cut-out.

7. A torsion spring based automatic injection device according to claim 5, wherein the cut-out comprises a distally located guiding surface extending substantially perpendicular to the longitudinal direction of the helical coiled torsion spring.

8. A torsion spring based automatic injection device according to claim 2, wherein the second surface comprises a step-wise configuration.
